(19) United States
(12) Patent Application Publication
(10) Pub. No.: US 2023/0010631 A1
(43) Pub. Date: Jan. 12, 2023

(54) KIT FOR A FIDUCIAL MARKER-BASED REGISTRATION OF PREINTERVENTIONAL IMAGE DATA TO AN INTRA-INTERVENTIONAL SCENE

(71) Applicant: Deutsches Krebsforschungszentrum, Heidelberg (DE)

(72) Inventors: Benjamin Johannes Mittmann, Heidelberg (DE); Alfred Franz, Heidelberg (DE); Alexander Seitel, Heidelberg (DE); Lena Maier-Hein, Heidelberg (DE)

(21) Appl. No.: 17/783,621

(22) PCT Filed: Dec. 8, 2020
(86) PCT No.: PCT/EP2020/085054

§ 371 Date: Jun. 8, 2022

(30) Foreign Application Priority Data
Dec. 9, 2019 (EP) 19214529.0

(51) Int. Cl. A61B 90/00; G06T 7/30; G06T 7/00

(52) U.S. Cl. CPC A61B 90/39; G06T 7/30; G06T 7/0012; G06T 2207/30204; G06T 2207/30088; A61B 2090/363

(57) ABSTRACT

A kit for a fiducial marker-based registration of preinterventional image data to an intra-interventional scene is disclosed. The kit comprises:
at least one first component, wherein the first component is configured for attachment to a body of a patient, wherein the first component comprises at least one base part of an arresting mechanism,
at least one second component, wherein the second component comprises at least one fiducial which is localizable in the preinterventional image data, wherein the second component comprises at least one first counterpart of the arresting mechanism,
at least one third component, wherein the third component comprises at least one mounting unit for mounting a spatially localizable tracking object on the third component, wherein the third component comprises at least one second counterpart of the arresting mechanism;
   wherein the second component and the third component are selectively attachable to the first component by establishing a releasable mechanical connection between the base part and the first counterpart or between the base part and the second counterpart.

### FIELD OF THE INVENTION

The invention relates to a kit for a fiducial marker-based registration of preinterventional image data to an intra-interventional scene and to a method for conducting a fiducial marker-based registration of preinterventional image data to an intra-interventional scene. The device and method of the present invention may mainly be used for a registration of computed tomography images or magnetic resonance imaging and ultrasonic images. The invention may specifically be applied to support medical interventions. Other applications may be feasible.

### RELATED WORK

Fiducial marker-based registration of preinterventional image data to an intra-interventional scene commonly plays an important role in the treatment of various diseases. Known methods for conducting a fiducial marker-based registration commonly comprise the following process steps. Before a planned medical intervention is conducted one or more fiducial markers are mounted onto a patient. Thereafter, preinterventional images such as computed tomography or magnetic resonance tomography images of the patient with the fiducial markers attached are acquired and the fiducial markers are localized in the preinterventional images. As an example, during the medical intervention, a spatial relation of computed tomography images or magnetic resonance images to the intra-interventional scene may be computed. This process may also be referred to as registration. A coordinate frame of the intra-interventional scene may be defined, for instance, by means of optical or electromagnetic tracking methods. With the tracking method, the spatial relation of another imaging modality, such as an ultrasound probe, to the intra-interventional scene may be measured in real-time. The positional information of the ultrasonic probe may be used to calculate a total transformation of a computed tomography images coordinate system or a magnetic resonance images coordinate system to an ultrasonic images coordinate system. This transformation may be applied to superimpose computed tomography images and ultrasonic images or magnetic resonance images and ultrasonic images. Directly after the medical intervention, the fiducial markers may be removed from the patient. However, other applications of fiducial markers are also possible.

In U.S. Pat. No. 9,572,539 B2 a device and a method for determining the position of an instrument with respect to medical images of a body are described. The device comprises a calibration support comprising a set of fiducial markers visible on medical images and fixed to the skin of a body, at least one micro-camera providing video images and mounted on the calibration support in a known position, and a processing unit connected to the micro-cameras and configured to process the video images from said micro-cameras to determine the position of in the instrument with respect to medical images.

In WO 2010/086374 A1 a method of navigation of a tool to be used in a surgical operation for treating a target region within the body of a patient is described. The method comprises the following steps for preparing the navigation: Disposing a first reference marker on the body of the patient, detecting the image position of said first reference marker by an image system, and detecting the spatial position of said first reference marker by a localizing system, calculating a first transform matrix between the image coordinate system of the imaging system and the spatial coordinate system associated with the first reference marker by using detected image and spatial positions of the first reference, in order to register data of the localizing and imaging systems together, detecting the spatial position of a pointer by the localizing system, constructing and displaying a navigation image, wherein a representation of the spatial position of the pointer is displayed on image data from the imaging system using the first transform matrix, determining an entry point through which the tool should be inserted for reaching the target region by using the representation of the pointer in the navigation image, disposing a second reference marker under sterile conditions on the body of the patient in the vicinity of the entry point, detecting the image position and spatial position of said second reference marker, calculating a second transform matrix between the image coordinate system of the imaging system and the spatial coordinate system associated with the second reference marker by using detected image and spatial positions of the second reference, in order to register data of the localizing and imaging systems together.

In WO 2016/059250 A1 a system for navigating a surgical instrument is described. The system comprises a processor configured for: Obtaining a first 3D medical image of a first volume of a patient's body, said first volume comprising a reference marker, registering the first 3D image with said reference marker, obtaining a second 3D medical image of a second volume of the patient's body, said second volume being different from the first volume and not containing the reference marker in its entirety, said first and second 3D images being obtained by a single imaging device, registering the second 3D medical image with the first 3D medical image, obtaining a virtual position of the surgical instrument with respect to the reference marker from a tracking system, determining a virtual position of the surgical instrument with respect to the second 3D medical image.

In WO 2016/059256 A1 a method for displaying the position and orientation of a linear instrument navigated with respect to a 3D medical image is described, wherein: said linear instrument is coupled to a guide; the guide is tracked by a navigation system with respect to the 3D image; a plane containing the axis of the linear instrument is virtually attached to the guide; a slice reformatted in the 3D image and containing said plane is displayed.

In U.S. Pat. No. 5,551,429 A a method for determining the location of the center of the imageable portion of a fiducial marker is disclosed. The method includes the use of a detachable cap. The lower portion of the cap has three arms and a boss for providing a detachable connection with an implanted base portion to which an imaging marker can be attached. The upper portion of the cap includes a divot-like depression that is configured to mate with a ball whose center can be determined. The ball, marker, and divot are configured so that the center of the ball, when mated to the divot, is coincident with the center of the marker when it is attached to the base in place of the cap. Knowledge of the location of the center of the ball when it is brought into engagement with the divot of the cap can be used to determine the location of the center of the marker when it is attached to the base.

In U.S. Pat. No. 6,073,044 A a detachable cap for use in determining the location of the center of the imageable portion of a fiducial marker is disclosed. The lower portion of the cap has three arms and a boss for providing a detachable connection with an implanted base portion to which an imaging marker can be attached. The upper portion of the cap includes a divot-like depression that is configured to mate with a ball whose center can be determined. The ball, marker, and divot are configured so that the center of the ball, when mated to the divot, is coincident with the center of the marker when it is attached to the base in place of the cap. Knowledge of the location of the center of the ball when it is brought into engagement with the divot of the cap can be used to determine the location of the center of the marker when it is attached to the base.

In U.S. Pat. No. 7,646,899 B2 a method for determining, an orientation of a base to which a fiducial marker is detachably mounted. The method includes the steps of determining the axis of symmetry for the fiducial marker and choosing the determined axis of symmetry of the fiducial marker as the axis of symmetry of the base.

In Xi Wen et al. (2014): “Automatic and Fast Registration Method for Image-Guided Surgery”, IEEE International Conference on Bioinformatics and Biomedicine, pages 597-600, a dynamic coordinate registration method is proposed. This method simplifies and automates the registration procedure between image space coordinates and patient space coordinates with a high accuracy. Compared with paired-point registration, three-dimensional registration can be performed with only one model, referred to as Navpass, instead of three or more points. The Navpass is a real-world model, which can be easily detected in computed tomography images. First, the pose information such as position and orientation of the Navpass is detected automatically on the images by a Dynamic Region Growing algorithm. Then, the In registration transformation is calculated using the corresponding location in the patient coordinates obtained by an electromagnetic tracking device in real time. In the navigation procedure, the method is capable of computing the registration error in real-time during the respiratory cycle and assisting the surgeon to insert needles within a minimal error. When the target registration error is beyond a certain threshold, the registration matrix would be updated automatically. The method has been tested on a real-world navigation system with a specially designed phantom. Based on the practical and extensive experiments, it is concluded that the method can provide fast and accurate registration with a 1.7±0.03 mm target registration error, and that it helps reduce the operation time dramatically for the surgeons as well as the patients.

In Filip Śuligoj et al. (2017): “Automated Marker Localization in the Planning Phase of Robotic Neurosurgery”, IEEE Access, Volume 5, pages 12265-12274, an automated marker localization in the planning phase of robotic neurosurgery is described. Accurate patient registration is a critical issue in medical image-guided interventions. The neurosurgical robotic system RObotic Neuro-NAvigation (RONNA) uses four retro-reflective spheres, on a marker attached to the patient's cranial bone, for patient registration in physical and image space. In this paper, the algorithm for automatic localization of spherical fiducials in CT scans is presented and clinically evaluated. The developed localization algorithm uses a unique approach, which combines machine vision algorithms, biomedical image filtration methods, and mathematical estimation methods. The performance of the localization algorithm was evaluated in comparison with four skilled human operators. The measurements were based on twelve patient and eight lab phantom CT scans. The localization error of the algorithm in comparison with the human readings was smaller by 49.29% according to the ground truth estimation and by 45.91% according to the intra-modal estimation. Localization processing time was reduced by 84.96%. Reliability in terms of successful localization of the fiducial marker was 100% for 20 different test samples containing a total of 116 spherical fiducials. Based on the tests carried out in clinical conditions, the localization algorithm has demonstrated reliability with a high degree of accuracy and short processing time. The developed algorithm provides fully automated and accurate machine vision-based patient localization for the neurosurgical clinical application of the robotic system RONNA.

In Krücker et al. (2007): “Electromagnetic Tracking for Thermal Ablation and BiopsyGuidance: Clinical Evaluation of Spatial Accuracy”, J Vasc Intery Radiol, Vol. 18(9), pages 1141-1150, the spatial accuracy of electromagnetic needle tracking was evaluated and the feasibility of ultrasonography (US)-computed tomography (CT) fusion during CT- and US guided biopsy and radiofrequency ablation procedures was demonstrated.

Further navigation trackers are described in two brochures provided by the company Stryker (Kalamazoo, Mich., USA), namely “CranialMap® 3.0 highlights summary” describing a navigation tracker on the market designed for sterile, soft tissue placement and “CranialMap® 3.0 Integrated navigation software solution” describing a simplified solution to incorporate navigation into neurosurgical procedures. From pre-operative planning to intra-operative guidance and control, CranialMap 3.0 has been designed to meet the demands in today's operating room and help deliver better patient outcomes. A further brochure describing navigation accessories is the Brainlab AG (Munich, Bavaria, Germany) “Product Catalogue Neurosurgery, November 2017, Rev. 3” presenting cranial accessories, IGS (image guided surgery) disposables and microscope navigation.

Despite the recent advances concerning devices and methods for fiducial marker-based registration of preinterventional image data to an intra-interventional scene there is still room for improvement. Common fiducial markers are often noninvasive markers which may be attachable to a skin site of the patient. However, noninvasive markers commonly prevent the possibility to conduct the preinterventional method steps, such as outlined above, one or two days before the actual medical intervention is conducted. The reason therefore is on the one hand that the noninvasive markers are dimensioned too large for the purpose to be mounted permanently onto the patient for a time period of several days. On the other hand, a removal of the markers from the skin site of the patient and a reattachment of the markers during the registration process is so far not possible since a reproducible retrieval of the original position of the markers on the patient after a removal of the markers cannot be guaranteed. Such a reproducible reattachment of the markers is so far only applicable when utilizing surgically anchored markers such as commonly applied in neurosurgery.

### Problem to be Solved

It is therefore an objective of the present invention to provide a kit for a fiducial marker-based registration of preinterventional image data to an intra-interventional scene and to provide a method for conducting a fiducial marker-based registration of preinterventional image data to an intra-interventional scene, which at least partially address the above-mentioned challenges and shortcomings of devices and methods of this kind. Specifically, the device and the method shall be patient-friendly and shall be integrable into a clinical routine in an easy manner.

### SUMMARY OF THE INVENTION

This problem is solved by a kit for a fiducial marker-based registration of preinterventional image data to an intra-interventional scene and by a method for conducting a fiducial marker-based registration of preinterventional image data to an intra-interventional scene having the features of the independent claims. Preferred embodiments of the invention, which may be realized in an isolated way or in any arbitrary combination, are disclosed in the dependent claims.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may all refer to a situation in which, besides B, no other element is present in A (i.e. a situation in which A solely and exclusively consists of B) and to a situation in which, besides B, one or more elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present invention, a kit for a fiducial marker-based registration of preinterventional image data to an intra-interventional scene is disclosed. The kit comprises in at least one first component. The first component is configured for attachment to a body of a patient. Further, the first component comprises at least one base part of an arresting mechanism. Further, the kit comprises at least one second component. The second component comprises at least one fiducial which is localizable in the preinterventional image data. Specifically, the second component may have a plurality, e.g, at least two, preferably at least three, of the fiducials. The second component comprises at least one first counterpart of the arresting mechanism. Further, the kit comprises at least one third component. The third component comprises at least one mounting unit for mounting a tracking object on the third component. Specifically, the mounting unit may be configured for permanently mounting a tracking object on the third component. The tracking object may be configured for determining a position of a medical device relative to the tracking object. Further, the tracking object may be spatially localizable. The third component comprises at least one second counterpart of the arresting mechanism. The second component and the third component are selectively attachable to the first component by establishing a releasable mechanical connection between the base part and the first counterpart or between the base part and the second counterpart. Each, the second component and the third component, may be detachable from the first component.

The term “preinterventional image data” as further used herein may refer to image data which is acquired prior to a medical intervention. The preinterventional image data specifically may comprise computed tomography images and/or magnetic resonance images. However, other kinds of images may be feasible. An acquisition of preinterventional image data may specifically not be feasible during the medical intervention. The term “medical intervention” may specifically refer to a surgical intervention or process, which uses operative manual and instrumental techniques on a patient to investigate or treat a pathological condition such as a disease or injury, to help improve bodily function or appearance or to repair unwanted ruptured areas. The term “medical intervention” may also be referred to as a “surgical procedure”, “operation” or “surgery”.

Thus, the kit may be configured in order to be suited for computed tomography images or magnetic resonance images. During image acquisition, a fiducial marker may be attached to the patient. Afterwards, the fiducial of the fiducial marker or a plurality of the fiducials of the fiducial marker may be localized in the computed tomography images or in the magnetic resonance images. During the medical intervention, a position of an ultrasonic probe relative to the fiducial marker may be calculable, specifically as a geometric distribution of the fiducial within the fiducial marker, specifically the second component, is known. Specifically, the position may be determinable via optical or electromagnetic tracking methods and via a calculation of a total transformation of a computed tomography images coordinate system in or magnetic resonance images coordinate system to an ultrasonic images coordinate system. Further, spatially superimposed images may be depictable. Directly after the medical intervention, the fiducial marker may be removed from the patient.

The term “patient” as further used herein may refer to a human being or an animal, independent from the fact that the human being or animal may be in a healthy condition or may suffer from one or more diseases. As an example, the patient may be a human being or an animal suffering from cancer. However, additionally or alternatively, the invention may be applied to other types of patients or diseases. The term “user” as further used herein may specifically refer to a physician or to a nurse. However, other users may be feasible.

As used herein, the term “ultrasonic probe” may generally refer to a component configured to transmit and/or receive ultrasound. An ultrasonic probe may also be referred to as an ultrasonic transducer. The ultrasonic probe may be configured to produce ultrasound, e.g. by converting electrical signals into ultrasound, or to convert ultrasound in electrical signals or both. Typically, ultrasonic probes, specifically ultrasonic probes used in medical diagnosis, may be classified as linear probes, sector probes and curved probes. The ultrasonic probe may further be combined or integrated with a light source, preferably a pulsed laser, configured to generate an optical signal, such as an electromagnetic wave in the optical spectrum that may be converted into an acoustic signal, specifically an ultrasonic signal, taking advantage of a photoacoustic effect. The ultrasonic probe may, in particular, be configured to receive the acoustic signal generated by the optical signal. A conversion from the optical to the acoustic signal taking advantage of the photoacoustic effect may, in particular, take place in the tissue of a patient. Thus, by receiving the acoustic signal the ultrasonic probe may be configured to measure additional properties of the tissue, typically optical or functional properties.

The term “kit” as further used herein refers to a group of at least two elements, which may interact in order to fulfill at least one common function. The at least two elements may be handled independently or may be coupled, connectable or integrable in order to form a common object. Thus, the kit generally refers to a group of at least two elements or components which are capable of interacting in order to perform at least one desired function, in the present case in order to perform a fiducial marker-based registration of preinterventional image data to an intra-interventional scene. The kit generally may also be referred to as a system or an assembly.

The term “fiducial marker” as further used herein refers to an object placed in a field of view of an imaging system which appears in an image produced, for use as a point or structure of reference or as a measure. It may be either something placed into or on the imaging subject, or a mark or a set of marks on another object, for instance in the reticle of an optical instrument.

The term “fiducial” as further used herein refers to an artificial, localizable structure of a fiducial marker. One fiducial or a plurality of fiducials may form part of a fiducial marker. Thus, a fiducial marker may comprise one or more of the fiducials. The fiducial marker may comprise at least one of the fiducials, preferably at least two of the fiducials, more preferably at least three of the fiducials, Specifically, the second component may be referred to as fiducial marker or the second component may comprise the fiducial marker.

The fiducial may specifically be made of a metal. Further, specifically, the fiducial may be a metal sphere. However, other embodiments may also be feasible. Specifically, a material of the fiducial may differ from a material of a basic body of the second component. Exemplarily, the fiducial may be made of a metal, specifically steel, and the basic body may be made of a polymer, specifically of a thermoplastic material.

A material of the fiducial may be selected from the group consisting of: a metal, specifically steel; a fluid, specifically an encapsulated fluid. Specifically, the fiducial may have a spherical shape. Specifically, the second component may comprise a plurality oldie fiducials and the fiducials may respectively have a spherical shape. The fiducial may have a diameter of 0.1 mm to 20 mm, preferably of 0.2 mm to 15 mm, preferably of 0.5 mm to 10 mm, most preferably of 5 mm. However, other shapes may be feasible. Exemplarily, the fiducial may be embodied as a stripe.

The fiducial marker may be used to conduct a point-based registration. The fiducial marker may be configured such that the at least one fiducial may be on the one hand localizable in computed tomography images and/or magnetic resonance images and may on the other hand enable a determination of a position of an ultrasonic probe relative to the fiducial marker via optical or electromagnetic tracking methods.

Further, the fiducial marker may comprise a scaffolding for enclosing the at least one fiducial. The scaffolding may exemplarily be made of a polymer material, specifically of a photopolymer. Thus, the second component may specifically be made of a polymer material, specifically of a photopolymer. Further, also the first component and/or the second component and/or the third component may be made of a polymer material, specifically of a photopolymer. However, other materials may also be feasible.

Specifically, a position of body structures of a patient may be defined by a so-called fiducial marker coordinate system. The fiducial marker coordinate system may be associated with the fiducial marker. Specifically, the second component may be configured for determining a transformation from an images coordinate system of the preinterventional images such as computed tomography images or magnetic resonance images into the fiducial marker coordinate system. The second component may be configured for defining an own coordinate system, specifically the fiducial marker coordinate system.

The fiducial marker coordinate system may be assigned to the fiducial marker. The fiducial marker coordinate system may be applicable to define positions of structures within the body of the patient.

The fiducial of the second component may be exchangeable. Specifically, the second component may comprise a plurality of the fiducials and each of the fiducials may be exchangeable. Exemplarily, the fiducial of the second component may be exchangeable in dependence on an imaging modality. For the purpose of exchanging the fiducials, the second component may be disassembled, the fiducial may be exchanged and thereafter, the second component may be assembled again. Thus, the second component may be suited universally for fiducial marker-based registration of different image modalities such as computed tomography, magnetic resonance imaging and positron emission tomography.

The term “registration” as further used herein refers to a transformation of coordinate systems defined by one or more image modalities such as computed tomography or magnetic resonance imaging and/or ultrasonic imaging into a predefined, common coordinate system. In the common coordinate system, the registered images as, for instance, computed tomography images and ultrasonic images or magnetic resonance images and ultrasonic images overlap spatially. As a consequence, helpful information from the computed tomography images or from the magnetic resonance images such as information on a position of critical structures in relation to the ultrasonic image plane or organ segmentations may be shown in addition to the ultrasonic image.

The term “fiducial marker-based registration” as further used herein refers to a registration, wherein a total transformation of a computed tomography images coordinate system or of a magnetic resonance images coordinate system into the ultrasonic images coordinate system comprises an application of a fiducial marker. Specifically, the total transformation of the computed tomography images coordinate system or of the magnetic resonance images coordinate system into the ultrasonic images coordinate system may comprise a cascade of single transformations, which are conducted one after the other. Thus, a first transformation may comprise a transformation of the computed tomography images coordinate system or of the magnetic resonance images coordinate system into a fiducial marker coordinate system. A second transformation may comprise a transformation of the fiducial marker coordinate system into a tracking coordinate system. A third transformation may comprise a transformation of the tracking coordinate system into an ultrasonic images coordinate system. Thus, the total transformation of a computed tomography images coordinate system or of a magnetic resonance images coordinate system into an ultrasonic images coordinate system may comprise at least three single transformations, preferably four single transformations, in total. Further, the fiducial marker-based registration may refer to a registration wherein at least two computed tomography images are merged. This process may be referred to as computed tomography to computed tomography registration. Moreover, the fiducial marker-based registration may refer to a registration wherein at least two magnetic resonance images are merged. Also other applications may be feasible.

The term “component” generally may refer to a part or an element of a system or an object, which may interact with another component of the system in order to fulfill at least one common function. The component may be handled independently to the other component or may be coupled, connectable or integrable to the other component in order to form a common object. The terms “first component”, “second component” and “third component” may be considered as description without specifying an order and without excluding a possibility that several kinds of first components, second components and third components may be applied. Further, additional components such as fourth components may be applied. As outlined above, the kit specifically may comprise at least one of the first components, at least one of the second components and at least one of the third components. Thus, the kit may comprise a plurality of the first components, a plurality of the second components and a plurality of the third components. However, preferably, the kit may comprise a single one of the first component, a single one of the second component and a single one of the third component.

Specifically, the base part may be firmly attached to the first component. Further, the first counterpart may be firmly attached to the second component. Moreover, the second counterpart may be firmly attached to the third component. As used herein, the term “firmly attached to” may generally refer to an object being stably connected to another object, for example via glue and/or an adhesive strip. The two objects may be detachable from one another, for example by means of an aid such as but not limited to a solvent. A detachment of the respective base part or counterpart from the respective component may render the respective component unserviceable. Specifically, the base part and the first component may form a unit. Thus, during the whole registration process, the base part may be attached to the in first component. After the registration process, the base part and the first component may be removed from the body of the patient as a whole.

Specifically, the first component may be a non-invasive component. Thus, the first component may be configured to be attached to the body of the patient without the necessity of a surgery procedure. Specifically, the first component may comprise at least one adhesive material attachable to a skin site of the patient such as a plaster. The first component may have a front side and an opposing rear side. The adhesive material may be arranged on the front side and the base part may be arranged on the rear side.

However, alternatively, the first component may be an invasive component. Thus, the first component may be configured to be attached to the body of the patient via a surgery procedure. Specifically, the first component may be configured to be attached to a bone of the patient via one or more screws.

The arresting mechanism may ensure a sufficiently high positional accuracy between the first component and the second component or between the first component and the third component. Thus, a fixed geometrical relation with regard to a spatial position and orientation between the second component and the third component is ensured. Based on the known fixed geometrical relation a constant transformation between the fiducial marker coordinate system and the coordinate system of the tracking object may be calculated. The constant transformation between the fiducial marker coordinate system and the tracking object system may already be calculable prior to conducting the registration process.

The term “arresting mechanism” may generally refer to an arbitrary element or to an arbitrary assembly of elements configured to fix or to partake in fixing an object in a certain position or to connect or to partake in connecting the object to another object. The arresting mechanism may achieve fixing an object in a certain position or connecting an object to another object on its own, e.g. as a clip, and/or in cooperation and/or interaction with another device or structure, e.g. another arresting mechanism. As used herein, the term “connectable” may generally refer to the property of an object that may be brought into and fixed in close physical proximity to another object. In particular, the object that is connectable to another object may be in direct or indirect physical contact with the other object when it is connected to it.

Each of the second component and the third component may be attachable to the first component in a predefined mounting position. As used herein, the term “predefined mounting position” may generally refer to a mounting position that had been chosen and/or determined as a possible mounting position before the object was actually mounted. The predefined mounting position may be marked and/or identified as such, specifically physically, e.g. by one or more of a protrusion, an elevation, an indentation and a depression. Specifically, the predefined mounting position may facilitate a mounting of an object, e.g. by identifying a location, in which the object may be mounted rapidly and/or easily and/or in which the object, when mounted, is particularly stable.

As generally used herein, the term “base part” refers to a component of an element which is configured to interact with a “counterpart” in order to form a connection or a connector. Thus, the base part and the counterpart may be complementary contours configured for forming a connection or, in conjunction, a connector. Therein, one of the base part or the counterpart may be or may comprise a male contour, such as a male plug, and the other one of the base part or the counterpart may be or may comprise a female contour, such as a female plug.

The base part may further be embodied as a body mount. As further used herein, the term “body mount” refers to a device which is attachable to the skin of the patient. Thus, the body mount may comprise at least one attachment component which is capable of connecting the body mount to the skin, such as at least one adhesive surface and/or at least one adhesive strip or plaster. The body mount may further comprise at least one body mount base, which may be used as a support for further objects, for attachment of further objects, such as the second component or the third component. Thus, generally, the body mount may also be referred to as a support. The first component may specifically be configured to stay on the body of the patient for at least 24 hours. Thus, an acquisition of preinterventional image data may be feasible at least one day before a surgery procedure is conducted.

Therein, generally, one or both of the base part and the counterpart involved may comprise at least one protrusion and, in a complementary fashion, the other one of the base part and the counterpart may comprise at least one groove or slot in which the protrusion may be guided to form the connection or connector. When releasing the connector or connection, the movements may be performed in the opposite order. Thus, as an example, a linear movement may be performed when attaching the base part and the counterpart and, subsequently, a linear movement may be performed, by pulling the base part and the counterpart apart along the axis.

Further, the base part and the counterpart may be formed by a single piece. As used herein the term “by a single piece” may generally refer to an element consisting of one component only or to an element consisting of at least two components which may be connected such that a disruption of the connection damages and/or destroys the element and/or renders the element unserviceable.

The terms “first counterpart” and “second counterpart” may be considered as description without specifying an order and without excluding a possibility that several kinds of first counterparts and second counterparts may be applied. Further, additional counterparts such as third counterparts may be applied.

The first counterpart and the second counterpart may respectively be formed such that the first counterpart and the second counterpart respectively geometrically tit to the base part. Exemplarily, the first counterpart and/or the second counterpart may comprise one or more guide pins. Specifically, the first counterpart and the second counterpart may be equally formed. The term “equally formed” specifically may refer to corresponding shapes of the first counterpart and the second counterpart specifically with regard to a geometry, a construction and a dimensioning. However, the second component and the third component may also be configured to be mountable on different locations of the first component.

As outlined above, the second component and the third component are selectively attachable to the first component. The term “selectively” may refer to a property of at least two components to be applicable independently from each other. Thus, the at least two components each may specifically be applicable alone, e.g. independently from each other. Specifically, either the second component or the third component may be attached to the first component.

As used herein, the term “mechanical connection” generally refers to a connection of two or more components by mechanical holding forces. As an example, the mechanical connection may be or may comprise at least one of a form-fit or a force-fit connection. In the case of the present arresting mechanism, specifically, the mechanical connection may be a form-fit connection. As further used herein, the term “releasable”, in the context of the mechanical connection, generally refers to the fact that the mechanical connection may be brought from a disconnected state, also referred to as a non-mated state, into a connected state, also referred to as a mated state, and back into the disconnected state. Thus, the mechanical connection may be closed and released at will. Specifically, the mechanical connection may be releasable without using any tools, simply by manual action. As an example, for detaching the base part and the counterpart from each other, forces of no more than 50 N, such as of no more than 20 N, such as of no more than 10 N, may be required, which may be applied by one hand or even the fingers or fingertips of the user.

Specifically, the arresting mechanism may comprise a snap-fit mechanism. The term snap-fit mechanism generally refers to an arbitrary assembly method used to attach at least two elements with each other in order to form an assembled unit, specifically by pushing inter-locking components of the elements together. There are a number of variations in snap-fits, including cantilever snap-fits, torsional snap-fits and annular snap-fits. Specifically, one of the base part and the counterpart may have a snap-in hook and the other one of the base part and the counterpart may have a protrusion. The snap-in hook may be configured to enclose the protrusion at least partially.

For further details with regard to the snap-fit mechanism, reference is made to the description of the figures below. Due to the snap-fit mechanism, a reproducible and high positional accuracy of the second component and the third component onto the first component is feasible. Deviations may be in the submillimeter range. Further, the snapping effect may give the user an indication that the second component and the third component are correctly mounted and fixed onto the first component.

Specifically, the base part may comprise at least one receptacle having a receptacle shape. Both, the first counterpart and the second counterpart, may comprise at least one protrusion having a protrusion shape, or vice versa. Thus, each of the first counterpart and the second counterpart may comprise the receptacle having the receptacle shape and the base part may comprise the protrusion having the protrusion shape. The protrusion shape may be complementary to the receptacle shape. The protrusion may be configured to be mounted into the receptacle such that a form-fit connection is formed. The first counterpart and the second counterpart may be configured to be led on or into the base part within a specified movement direction, particularly a linear movement direction. Specifically, the base part or both, the first counterpart and the second counterpart, may have at least two outer protrusions and at least one intermediate protrusion arranged between the at least two outer protrusions. The intermediate protrusion may have at least one snap-in hook. Each of the two outer protrusions may have a length lₒₚ. The intermediate protrusion may have a length lᵢₚ. The length lₒₚ may be shorter than the length lᵢₚ.

Specifically, the arresting mechanism may comprise a sliding guide, specifically a dovetail joint. The term “dovetail joint” may generally refer to a joinery technique most commonly wherein at least one pin cut to extend from one end of one component is configured to inter-lock with at least one tail cut into the end of another component. The pin and the tail may have a trapezoidal shape. Specifically, the base part may have the at least one dovetail guide. The dovetail guide may be located at at least one first end wall of the first component. Alternatively, each of the second component and the third component may have the dovetail guide. Specifically, the dovetail guide may be located at at least one second end wall of the first counterpart and of the second counterpart. For further details with regard to the dovetail in joint, reference is made to the description of the figures below.

Further, the arresting mechanism may comprise at least one locking mechanism. The locking mechanism may be configured to be manually activated and deactivated by a user. Specifically, the locking mechanism may comprise at least one lever. The lever may be configured to be manually switched by the user. Due to the locking mechanism, a risk of undesired disassembly of the first component and the second component or of the first component and the third component while the first component is attached to the body of the patient is generally reduced. As used herein, the term “lever” may generally refer to a mechanical element comprising an elongated arm, also referred to as a lever arm, which is pivotably mounted to at least one hinge or fulcrum. Specifically, the lever may be pivotably attached to one end of the first component. The lever may be permanently mounted to the first component. Still, other embodiments are feasible.

The locking mechanism specifically may be or may comprise a self-locking mechanism. As used herein, a “self-locking mechanism” generally refers to a mechanism which has at least one locked state and at least one unlocked state, wherein the mechanism, once brought into the locked state, is configured to maintain the locked state, such as by exerting forces onto at least one locking element of the self-locking mechanism in order to maintain this locking element in the locked position or locked state.

Both, the second component and the third component, may be configured to be attachable to the first component by moving the second component and the third component parallel to a skin site of the patient. Thus, for the purpose of attaching or detaching the second component or the third component to or from the first component, only little pressure onto the skin of the patient has to be applied by the user. Further, by moving the second component and the third component parallel to the skin site of the patient, shear forces in the skin region around the first component may be minimized. However, the second component and the third component, may, additionally or alternatively, be configured to be attachable to the first component by a rotating movement and/or a screwing movement. Also other kinds of movement may be feasible.

The first component having the base part of the arresting mechanism specifically may be configured as a flat element, wherein a length of the first component may exceed a thickness of the first component at least by a factor of 2, preferably by at least a factor of 5, more preferably by at least a factor of 10. Thus, a tilting of the first component when mounted on the body of the user may be reduced.

As outlined above, the kit comprises the second component. The second component may be configured for being attached to the first component before preinterventional image data is acquired. After the acquisition of preinterventional image data the second component may be removed from the first component while the first component stays onto the body of the patient. As the second component is configured for being attached to the first component only during the process of acquiring preinterventional image data, there are generally no restrictions with regard to dimensions of the second component. In case of magnetic resonance tomography images, small dimensions of the second component are preferred.

The term “localizable in the preinterventional image data” may specifically refer to a property of the fiducial of being visible in the preinterventional image data.

Specifically, the second component may comprise at least one first portion and at least one second portion. The first portion and the second portion may be reversibly assembled to each other. The first portion may comprise at least one first half of a receptacle for the fiducial, specifically for at least one of the fiducials, and the second portion may comprise at least one second half of the receptacle for the fiducial, specifically for at least one of the fiducials. Specifically, a number of receptacles may be equal to a number of fiducials of the second component. Specifically, one of the receptacles may be configured for receiving one fiducial. The fiducials may be exchangeable by disassembling the first portion and the second portion. Specifically, the receptacle may protrude from an outer surface of the second component. The second component may comprise a plurality of the fiducials and the fiducials may be located on different levels of the second component.

The third component may be configured for holding a tracking object. Specifically, the third component, specifically the mounting unit, may be configured for ensuring a predefined position and orientation of the tracking object, specifically when the tracking object is mounted on the third component. For this purpose, the third component may have the mounting unit. The third component may be configured for being mounted onto the first component prior to conducting a medical intervention and may stay onto the first component while the medical intervention is conducted. During the acquisition of the preinterventional image data, the third component may not be mounted onto the first component.

The term “mounting unit” may generally refer to a component configured to partake in connecting, at least two objects A and B. Thus, the mounting unit may also be referred to as a connection element. When connected, the two objects A and B may not be connected to one another directly and/or may not be in direct physical contact with one another. Instead, the two objects may be connected via at least one connection element and/or the two objects may each be in direct physical contact with the connection element, wherein the connection element the object A is connected to may differ from the connection element object B is connected to. Specifically, the mounting unit may partake in connecting at least two objects A and B by being itself connected to at least two components of the connection, Specifically, the mounting unit for the tracking object may comprise a screw connection. However, other embodiments may be feasible. Thus, the tracking object does not necessarily have to be mounted onto the body of the patient in a permanent manner. Further, the tracking object may be exchangeable easily.

As outlined above, the mounting unit may be configured for permanently mounting the tracking object on the third component. Thus, the tracking object may be fixable to the third component in such a manner that a reciprocal dislocation is prevented, also in case of a tilting of these elements. Specifically, the tracking object may stay on the third component also without a power transmission or at an interrupted power transmission.

The tracking object may optionally be part of the kit. Thus, the kit may further comprise the tracking object.

The tracking object may be selected from the group consisting of: an electromagnetic sensor; an electromagnetic field generator; a transponder; a sensor array; an optical sensor, specifically a camera, more specifically a micro-camera; active optical markers of optical tracking systems; passive optical markers of optical tracking systems; connectors for mechanical tracking stereotactic devices, inertial tracking systems, specifically inertial measurement units, ultrasound-based tracking devices, radio-frequency identification system. Also other embodiments may be feasible. The tracking object may define an own coordinate system. The coordinate system may be referred to as tracking object coordinate system.

As used herein, the term “electromagnetic field generator” may generally refer to an arbitrary component configured to produce an electric and/or magnetic field, in which a sensor array may be localized spatially. As used herein, the term “sensor array” may generally refer to a device configured to receive an electric and/or magnetic signal. Specifically, the electric and/or magnetic signal received by the sensor array may comprise information such as but not limited to a position of the sensor array or an orientation of the sensor array. The electric and/or magnetic signal may in particular be emitted by the electromagnetic field generator. Exemplarily, an object may be localized spatially with the aid of the sensor array, by mounting the sensor array rigidly on the object.

In a further aspect of the present invention a method for conducting a fiducial marker-based registration of preinterventional image data to an intra-interventional scene is disclosed.

The method comprises the method steps as given in the independent claims and as listed as follows. The method steps may be performed in the given order. However, other orders of the method steps are feasible. Further, one or more of the method steps may be performed in parallel and/or in a timely overlapping fashion. Further, one or more of the method steps may be performed repeatedly. Further, additional method steps may be present, which are not listed.

The method comprises the following steps:
a) providing at least one kit as described above or as will further be described below in more detail;
b) attaching the first component to the body of the patient;
c) attaching the second component to the first component by establishing the releasable mechanical connection between the base part and the first counterpart;
d) acquiring preinterventional image data via the first component and the second component and localizing the fiducial of the second component in the preinterventional image data;
e) removing the second component from the first component by releasing the mechanical connection between the base part and the first counterpart;
f) mounting the tracking object onto the third component via the mounting unit;
g) attaching the third component to the first component by establishing a releasable mechanical connection between the base part and the second counterpart;
h) using information obtained from the first component, the second component and the third component and information obtained from medical imaging to generate supportive information for physicians during a medical intervention;
i) removing the third component from the first component by releasing the mechanical connection between the base part and the second counterpart.

Specifically, the second component may comprise a plurality of the fiducials and step d) may comprise localizing the plurality of the fiducials of the second component in the preinterventional image data. Step d) may specifically be conducted by applying the fiducial marker of the second component and localizing of the fiducial marker in the preinterventional image data. Step h) may comprise determining a position of a medical device relative to the tracking object thereby establishing the intra-interventional scene. During step h) the transformation from the computed tomography images coordinate system or the magnetic resonance images coordinate system to the ultrasonic images coordinate system may be calculated. The intra-interventional scene may comprise a live visualization of spatial superimposed images.

In a further aspect, a further kit for a fiducial marker-based registration of preinterventional image data to an intra-interventional scene is described. The further kit comprises the third component. The third component corresponds to the third component of the kit as described above or as will further be described below in more detail. Thus, reference is made to the description of the third component above and to the description of the figures below.

Moreover, the further kit comprises at least one further component. The further component comprises the features and properties of the first component and the second component as described above or as will further be described below in more detail. Thus, reference is made to the description of the first component and the second component above and to the description of the figures below. The further component is configured for attachment to a body of a patient. In addition, the further component comprises at least one fiducial which is localizable in the preinterventional image data. Moreover, the further component comprises at least one base part of an arresting mechanism. The third component is attachable to the further component by establishing a releasable mechanical connection between the base part and the counterpart.

The proposed kit and the proposed method provide many advantages over known devices and methods.

Commonly, a fiducial marker is to be used as only one single unit. Thus, the fiducial marker is commonly mounted onto the patient at the beginning of the registration process and is removed as a whole after the registration process is finished. However, during the registration process the fiducial marker, specifically the fiducial, may not be removed from the patient and its configuration may not be changed. Thus, it is commonly not possible to disassemble the fiducial marker beforehand in diverse components to apply these components selectively and independently from one another during the registration process.

To the contrary, the kit according to the present invention specifically may comprise a division into three different and separate components, which may fulfill all functional tasks as a common device as a whole.

The acquisition of the preinterventional image data does not necessarily have to be conducted on the same day or shortly before the medical intervention. The acquisition of the preinterventional image data may also be conducted one or two days before the medical intervention is conducted.

The system may be suitable for electromagnetic tracking objects as well as for optical tracking objects. For the purpose of changing the tracking object only the mounting unit has to be modified and a constant transformation of the fiducial marker coordinate system to the tracking object coordinate system has to be adapted.

The kit is integrable into a clinical process in an easy manner. A mounting of the first component onto the body of the patient only requires little time such as the attaching and detaching of the second component and the third component to and from the first component.

Further, a highly reproducible positional accuracy is feasible when utilizing a non-invasive first component.

Further, through the fixed geometrical relation between the second component and the third component a manual touching of landmarks on the fiducial marker with a tracked pointer can be omitted in order to preserve a transformation from the fiducial marker coordinate system to the tracking coordinate system. This process is commonly time consuming and, thus, commonly not easily integrable into a clinical routine.

Summarizing the findings of the present invention, the following embodiments are preferred:

Embodiment 1: A kit for a fiducial marker-based registration of pre-interventional image data to an intra-interventional scene, the kit comprising:
at least one first component, wherein the first component is configured for attachment to a body of a patient, wherein the first component comprises at least one base part of an arresting mechanism,
at least one second component, wherein the second component comprises at least one fiducial which is localizable in the preinterventional image data, wherein the second component comprises at least one first counterpart of the arresting mechanism,
at least one third component, wherein the third component comprises at least one mounting unit for mounting a spatially localizable tracking object on the third component, wherein the third component comprises at least one second counterpart of the arresting mechanism;
wherein the second component and the third component are selectively attachable to the first component by establishing a releasable mechanical connection between the base part and the first counterpart or between the base part and the second counterpart.

Embodiment 2: The kit according to the preceding embodiment, wherein the first component is a non-invasive component.

Embodiment 3: The kit according to any one of the preceding embodiments, wherein the first component comprises at least one adhesive material attachable to a skin site of the patient.

Embodiment 4: The kit according to the preceding embodiment, wherein the first component has a front side and an opposing rear side, wherein the adhesive material is arranged on the front side, wherein the base part is arranged on the rear side.

Embodiment 5: The kit according to any one of the preceding embodiments, wherein the first component is an invasive component.

Embodiment 6: The kit according to any one of the preceding embodiments, wherein the kit comprises a single one of the first component, a single one of the second component and a single one of the third component.

Embodiment 7: The kit according to any one of the preceding embodiments, wherein the arresting mechanism comprises a snap-fit mechanism.

Embodiment 8: The kit according to any one of the preceding embodiments, wherein the base part comprises at least one receptacle having a receptacle shape, wherein the first counterpart and the second counterpart comprise at least one protrusion having a protrusion shape, wherein the protrusion shape is complementary to the receptacle shape, wherein the protrusion is configured to be mounted into the receptacle such that a form-fit connection is formed.

Embodiment 9: The kit according to any one of the preceding embodiments, wherein the arresting mechanism comprises a sliding guide, specifically a dovetail joint.

Embodiment 10: The kit according to any one of the preceding embodiments, wherein the arresting mechanism comprises at least one locking mechanism, wherein the locking mechanism is configured to be manually activated and deactivated by a user.

Embodiment 11: The kit according to the preceding embodiment, wherein the locking mechanism comprises at least one lever, wherein the lever is configured to be manually switched by the user.

Embodiment 12: The kit according to any one of the preceding embodiments, wherein both, the second component and the third component, are configured to be attachable to the first component by moving the second component and the third component parallel to a skin site of the patient.

Embodiment 13: The kit according to any one of the preceding embodiments, wherein the first component is configured to stay on the body of the patient for at least 24 hours.

Embodiment 14: The kit according to any one of the preceding embodiments, wherein the fiducial is selected from the group consisting of: a metal, specifically steel; a fluid, specifically an encapsulated fluid.

Embodiment 15: The kit according to any one of the preceding embodiments, wherein the fiducial has a spherical shape.

Embodiment 16: The kit according to the preceding embodiment, wherein the fiducial has a diameter of 0.1 mm to 20 mm, preferably of 0.2 mm to 15 mm, preferably of 0.5 mm to 10 mm, most preferably of 5 mm.

Embodiment 17: The kit according to any one of the preceding embodiments, wherein the fiducial of the second component is exchangeable.

Embodiment 18: The kit according to any one of the preceding embodiments, wherein the second component comprises at least one first portion and at least one second portion, wherein the first portion and the second portion are reversibly assemblable to each other, wherein the first portion comprises at least one first half of a receptacle for the fiducial, wherein the second portion comprises at least one second half of the receptacle for the fiducial, wherein the fiducial is exchangeable by disassembling the first portion and the second portion.

Embodiment 19: The kit according to the preceding embodiment, wherein the receptacle protrudes from an outer surface of the second component.

Embodiment 20: The kit according to any one of the preceding embodiments, wherein the second component comprises a plurality of fiducials, wherein the fiducials are located on different levels of the second component.

Embodiment 21: The kit according to any one of the preceding embodiments, wherein the mounting unit for the tracking object comprises a screw connection.

Embodiment 22: The kit according to any one of the preceding embodiments, wherein the kit further comprises the tracking object.

Embodiment 23: The kit according to the preceding embodiment, wherein the tracking object is selected from the group consisting of: an electromagnetic sensor; an electromagnetic field generator; a transponder; a sensor array; an optical sensor, specifically a camera, more specifically a micro-camera; active optical markers of optical tracking systems; passive optical markers of optical tracking systems; connectors for mechanical tracking; stereotactic devices; inertial tracking systems, specifically inertial measurement units; ultrasound-based tracking devices; radio-frequency identification system.

Embodiment 24: A method for conducting a fiducial marker-based registration of preinterventional image data to an intra-interventional scene, the method comprising the following steps:
a) providing at least one kit according to any one of the preceding claims referring to a kit;
b) attaching the first component to the body of the patient:
c) attaching the second component to the first component by establishing the releasable mechanical connection between the base part and the first counterpart;
d) acquiring preinterventional image data via the first component and the second component and localizing the fiducial of the second component in the preinterventional image data;
e) removing the second component from the first component by releasing the mechanical connection between the base part and the first counterpart;
f) mounting the tracking object onto the third component via the mounting unit;
g) attaching the third component to the first component by establishing a releasable mechanical connection between the base part and the second counterpart;
h) using information obtained from the first component, the second component and the third component and information obtained from medical imaging to generate supportive information for physicians during a medical intervention;
i) removing the third component from the first component by releasing the mechanical connection between the base part and the second counterpart.

### SHORT DESCRIPTION OF THE FIGURES

Further details of the invention may be derived from the following disclosure of preferred embodiments. The features of the embodiments may be realized in an isolated way or in any combination. The invention is not restricted to the embodiments. The embodiments are schematically depicted in the figures. Identical reference numbers in the figures refer to identical elements or functionally identical elements or elements corresponding to each other with regard to their functions.

In the Figures:

FIGS. 1A to 1B show an exemplary embodiment of a kit according to the present invention in two different perspective views;

FIGS. 2A to 2D show an exemplary embodiment of a first component of a kit according to the present invention in perspective views (FIGS. 2A, 2C, 2D) and in a cross-section view (FIG. 2B);

FIGS. 3A to 3D show an exemplary embodiment of a third component of a kit according to the present invention in different perspective views;

FIGS. 4A to 4B show a first portion of an exemplary embodiment of a second component of a kit according to the present invention in different perspective views; and

FIGS. 5A to 5B show a second portion of an exemplary embodiment of a second component of a kit according to the present invention in different perspective views.

These Figures and the various features comprised therein will be elucidated in a combined fashion below.

### DETAILED DESCRIPTION OF THE EMBODIMENTS

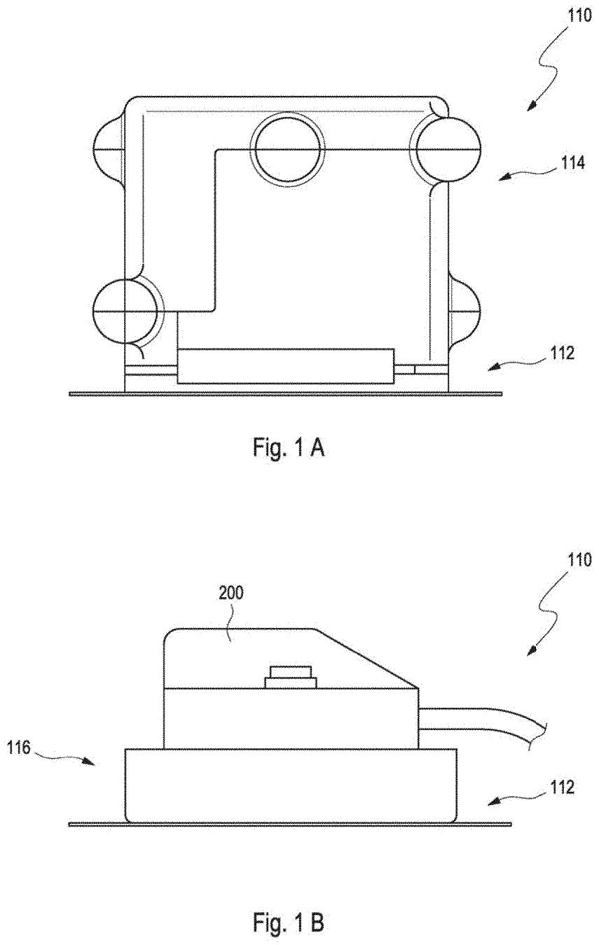

FIGS. 1A to 1B show an exemplary embodiment of a kit 110 according to the present invention in two different perspective views.

The kit 110 comprises at least one first component 112 configured for attachment to a body of a patient. Further, the kit 110 comprises at least one second component 114 and at least one third component 116. The second component 114 and the third component 116 are selectively attachable to the first component 112. Thus, the second component 114 is attachable to the first component 112. This scenario is illustrated in FIG. 1A. Alternatively, the third component 116 is attachable to the first component 112. This scenario is illustrated in FIG. 1B. A tracking object 200 may be mounted onto the third component 116.

Further details of the first component 112, the second component 114 and the third component 116 are given in the subsequent Figures. Thus, reference is made to the description below.

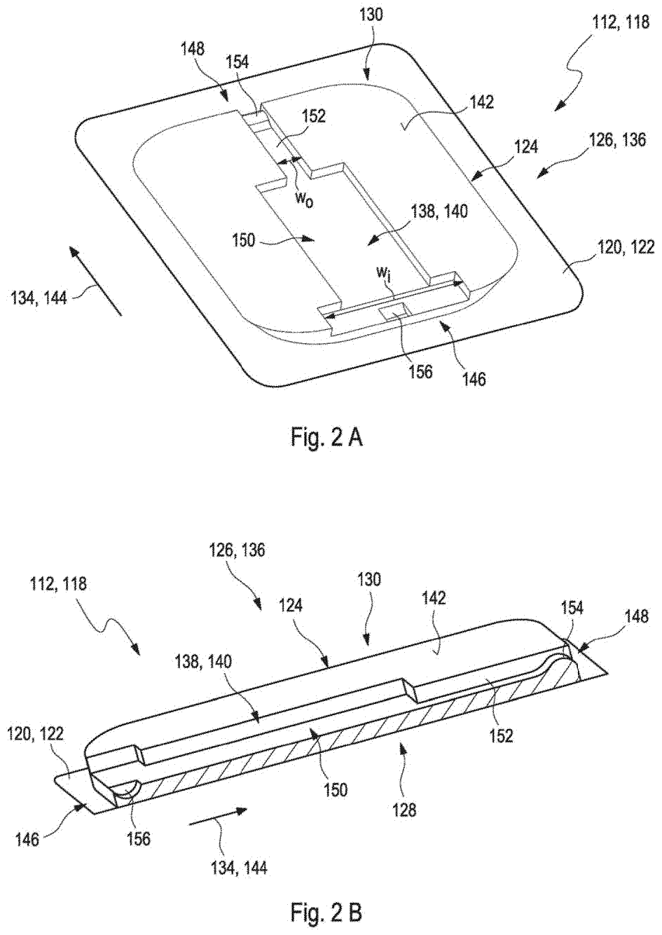

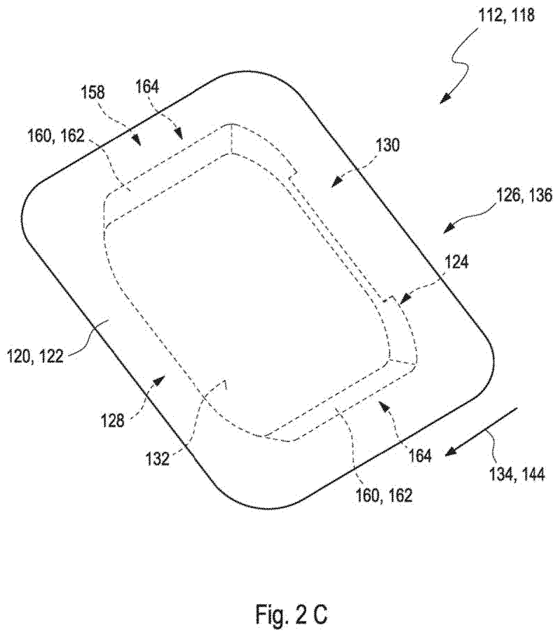

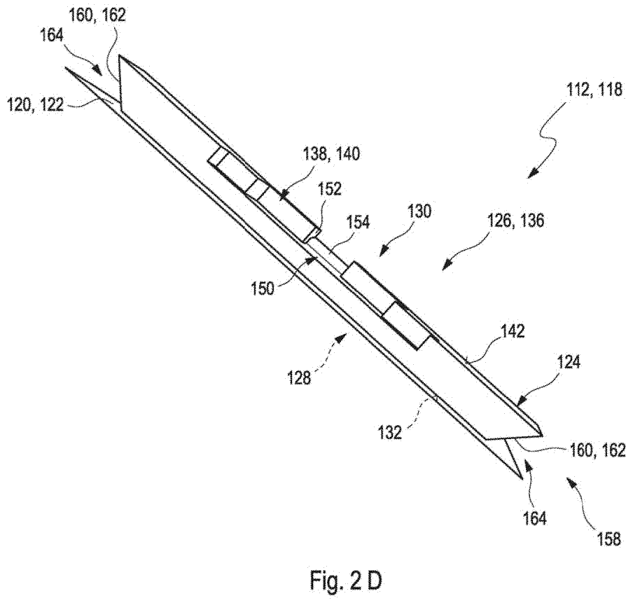

FIGS. 2A to 2D show an exemplary embodiment of a first component of a kit according to the present invention in perspective views (FIGS. 2A, 2C, 2D) and in a cross-section view (FIG. 2B);

The first component 112 is configured for attachment to a body of a patient. Specifically, the first component may be a non-invasive component 118. Thus, the first component 112 may have at least one adhesive material 120 such as a plaster 122. The first component 112 may specifically be configured to stay on the body of the patient for at least 24 hours. The first component 112 has a base part 124 of an arresting mechanism 126 as will further be described below. The first component 112 may have a front side 128 and an opposing rear side 130. The adhesive material 120 may be arranged on the front side 128 and the base part 124 may be arranged on the rear side 130. As illustrated in FIG. 2C, the front side 128 may comprise a flat surface 132 configured for attachment of the adhesive material 120.

Both, the second component 114 and the third component 116, may be configured to be mounted on the first component 112 by performing a linear movement in a direction parallel to a direction of extension 134 of the first component 112. The direction of extension 134 may thus be equivalent to a sliding direction 144 of the second component 114 and of the third component 116 relative to the first component 112.

The arresting mechanism 126 may comprise a snap-fit mechanism 136. Specifically, the base part 124 of the arresting mechanism may comprise at least one receptacle 138 having a receptacle shape 140. The receptacle 138 may extend over a whole surface of the first component 112. The receptacle 138 may extend along the sliding direction 144. As illustrated in FIG. 2A, the receptacle 138 may have an introducing end 146 from which a counterpart of the base part 124 can be threaded into the receptacle 138. An opposing end 148 may have a width wₒ being smaller than a width wᵢ of the introducing end 146. Specifically, the receptacle 138 may have a square basic shape. Thus, the receptacle 138 may have a rectangular receptacle 150 with a neighboring channel-shaped receptacle 152. As can specifically be seen in the cross-sectional view of FIG. 2B, the first component 112 may have at least one protrusion 154. The protrusion 154 may be configured to be enclosed by a snap-in hook as will further be described below in more detail. The protrusion 154 may be located at the opposing end 148 of the receptacle 138. Further, the introducing end 146 may comprise at least one receptacle 156.

The arresting mechanism 126 may further comprise a dovetail joint 158. This is specifically illustrated in FIGS. 2C and 2D. Specifically, the base part 124 may have at least one dovetail guide 160 with at least one longitudinal protrusion 162. The longitudinal protrusion 162 may have a trapezoidal shape. Specifically, the dovetail guide 160 may be located at one first end wall 164 of the first component 112 being parallel to a sliding direction of the second component 114 and the third component 116.

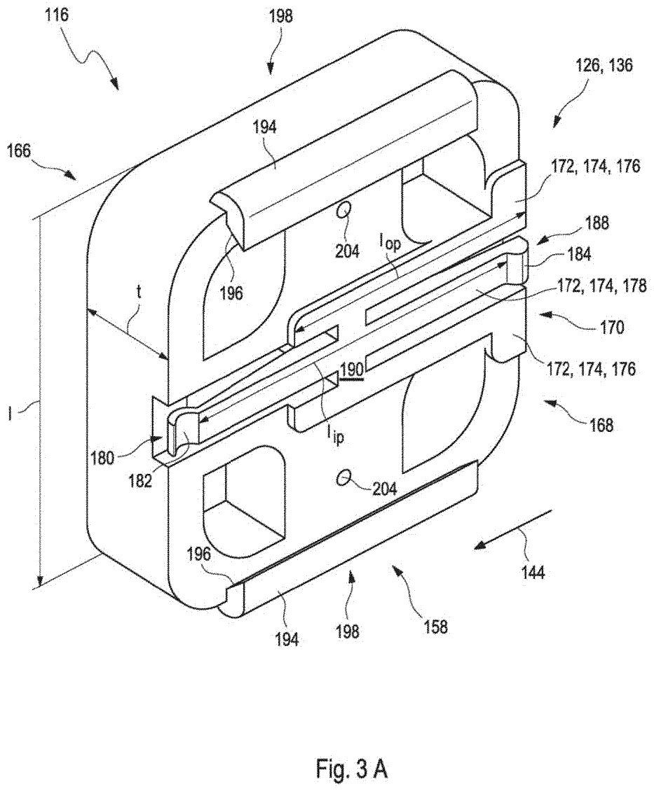

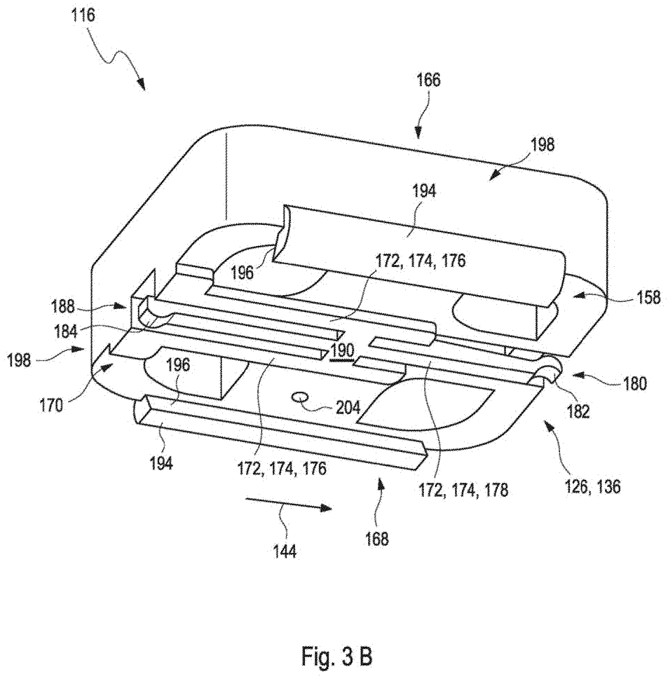

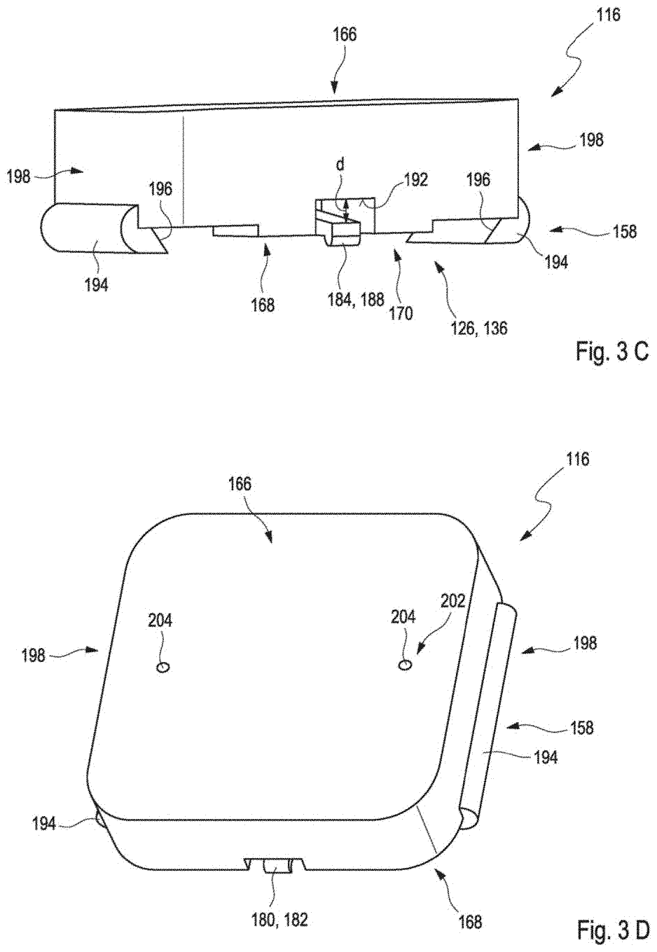

FIGS. 3A to 3D show an exemplary embodiment of a third component 116 of a kit 110 according to the present invention in different perspective views.

The third component 116 may exemplarily have a rectangular ground shape. Specially, the third component 116 may have a flat shape wherein a length I exceeds a thickness t at least by a factor of 2. The third component 116 may have a front side 166 and an opposing rear side 168.

A more detailed view of the rear side 168 is illustrated in FIGS. 3A to 3C. The third component 116 is attachable to the first component 112 such as illustrated in FIG. 2A to 2D. Thus, reference is made to description of FIGS. 2A to 2D above. The third component 116 comprises at least one second counterpart 170 of the arresting mechanism 126. Specifically, the second counterpart 170 may be located on the rear side 168.

As outlined above with regard to the first component as illustrated in FIGS. 2A to 2D, the arresting mechanism 126 comprises the snap-fit mechanism 136. Specifically, the second counterpart 170 may comprise at least one protrusion 172 having a protrusion shape 174. Specifically, the second counterpart 170 may comprise three of the protrusions 172, two outer protrusions 176 and one intermediate protrusion 178. The intermediate protrusion 178 may extend along a whole surface of the third component 116. The intermediate protrusion 178 may extend along the sliding direction 144. As illustrated in FIG. 3A, the intermediate protrusion 178 may have an introducing intermediate protrusion end 180 from which the second counterpart 170 can be threaded into the receptacle 138 of the first component 112 as illustrated in FIGS. 2A to 2D. The introducing intermediate protrusion end 180 may have a snap-in hook 182. The snap-in hook 182 may be configured to enclose the protrusion 154 of the base part 124 of the first component 112 as illustrated in FIGS. 2A to 2D. Further, the intermediate protrusion 178 may have at least one protrusion 184 located at an opposing end 188 of the introducing intermediate protrusion end 180. The protrusion 184 may be configured to be received in the receptacle 156 of the base part 124 of the first component 112 as illustrated in FIGS. 2A to 2D. The two outer protrusions 176 may have a length lₒₚ which is shorter than a length lᵢₚ of the intermediate protrusion 178. Each of the two outer protrusions 176 may be fixedly connected to the third component 116. To the contrary, the intermediate protrusion 178 may be fixedly connected to the third component 116 in a middle section 190 of the intermediate protrusion 178. As specifically illustrated in FIG. 3C, the opposing end 188 may have a distance d to a surface 192 of the third component 116. Thus, the protrusion 184 may have play to snap into the receptacle 156 of the base part 124 of the first component 112 as illustrated in FIGS. 2A to 2D. Accordingly, the snap-in hook 182 has a distanced to a surface 192 of the third component 116. Thus, snap-in hook 182 may have play to enclose the protrusion 154 of the base part 124 of the first component 112 as illustrated in FIGS. 2A to 2D.

As outlined above with regard to FIGS. 2A to 2D, the arresting mechanism 126 may further comprise the dovetail joint 158. This is specifically illustrated in FIGS. 3A to 3C. Specifically, the second counterpart 170 may have at least one dovetail groove 194 with at least one longitudinal slot 196. The longitudinal slot 196 may have a trapezoidal shape. Specifically, the dovetail groove 194 may be located at one second end wall 198 of the third component 116 being parallel to the sliding direction 144 of the third component 116.

In FIG. 3D, the front side 166 of the third component 116 is illustrated. The third component 116 may comprise a mounting unit 202 for the tracking object 200, such as illustrated in FIG. 1B. The mounting unit 202 may exemplarily comprise holes 204 for screws. Other embodiments may be feasible.

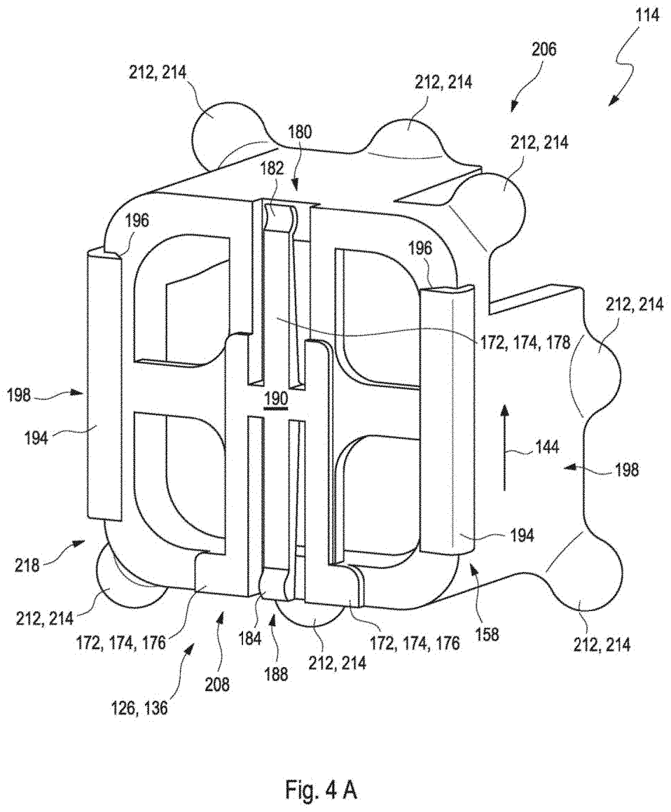

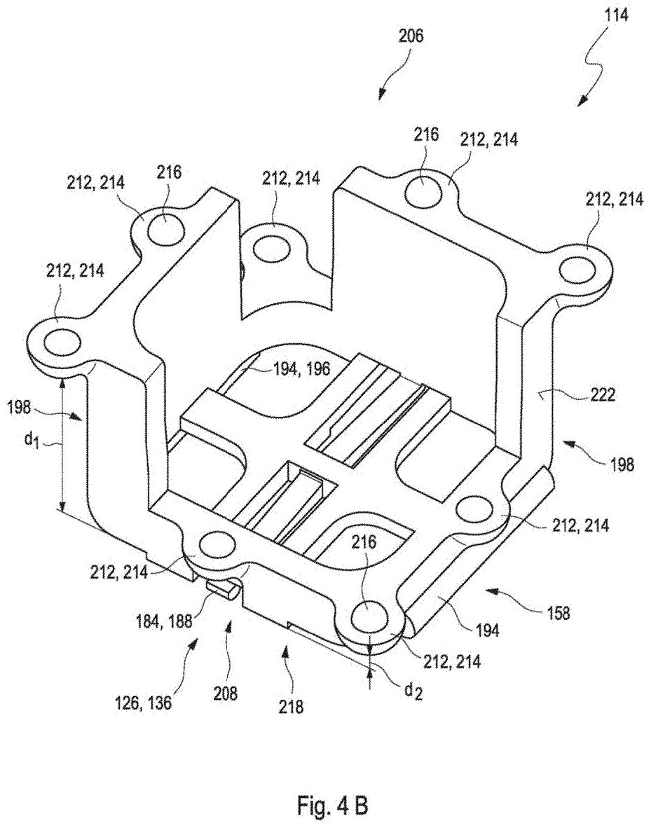

FIGS. 4A to 4B show a first portion 206 of an exemplary embodiment of a second component 114 of a kit 110 according to the present invention in different perspective views.

The first portion 206 comprises a first counterpart 208 of the arresting mechanism 126. The first counterpart 208 and the second counterpart 170 may be equally formed. Thus, with regard to the first counterpart 208 reference can be made to the description of FIGS. 3A to 3D above.

The arresting mechanism 126 comprises the snap-fit mechanism 136. Specifically, the first counterpart 208 may have at least one protrusion 172 having the protrusion shape 174. Specifically, the first counterpart 208 may comprise three of the protrusions 172, the two outer protrusions 176 and the one intermediate protrusion 178.

As outlined above with regard to FIGS. 2A to 2D, the arresting mechanism 126 may further comprise the dovetail joint 158. This is specifically illustrated in FIGS. 3A to 3C. Specifically, the second counterpart 170 may have at least one dovetail groove 194 with at least one longitudinal slot 196. The longitudinal slot 196 may have a trapezoidal shape. Specifically, the dovetail groove 194 may be located at one second end wall 198 of the third component 116 being parallel to the sliding direction 144 of the third component 116. Further, the arresting mechanism 126 may further comprise the dovetail joint 158. Specifically, the first counterpart 208 may have the at least one dovetail groove 194 with the at least one longitudinal slot 196.

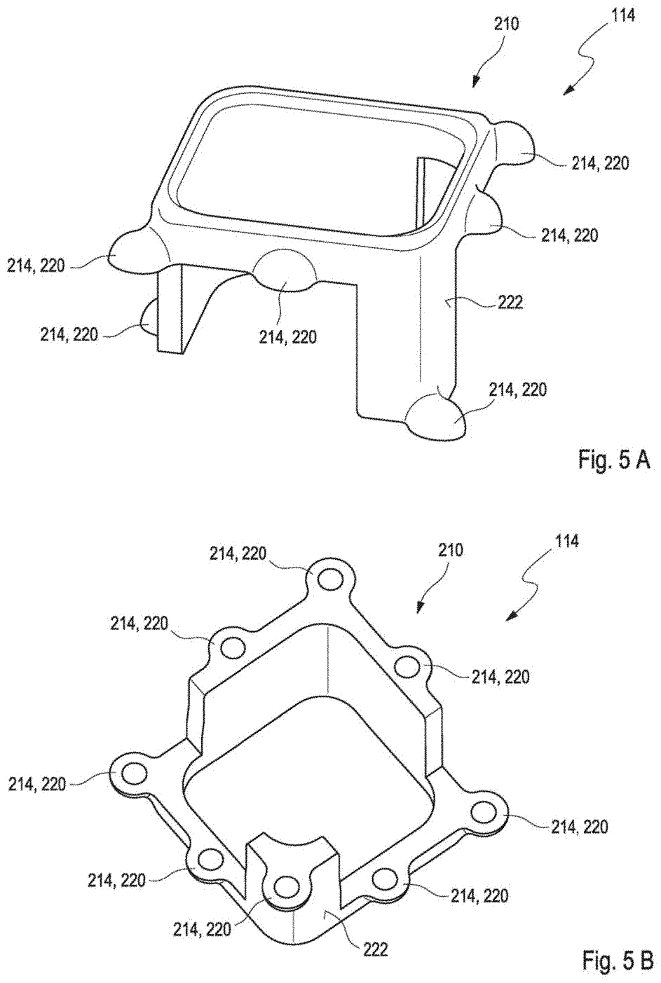

The first portion 206 may be connectable to a second portion 210 which is exemplarily illustrated in FIGS. 5A and 5B. The first portion 206 may comprise at least one first half 212 of a receptacle 214 for one of the fiducials 216. Exemplarily, the fiducials 216 may have a spherical shape and may be made of steel. The fiducials 216 may be located on different levels of the second component 114. Thus, the receptacles 214 may have different distances d to a rear side 218 of the second component 114. As exemplarily illustrated in FIG. 4B, some of the first halves 212 may have a distance d₁ to the rear side 218 and some of the first halves 212 may have a distance d₂ to the rear side 218 wherein the distance d₁ is larger than the distance d₂.

The first portion 206 and the second portion 210 may be reversibly assemblable to each other. The fiducials 216 may be exchangeable by disassembling the first portion 206 and the second portion 210.

FIGS. 5A to 5B show a second portion 210 of an exemplarily embodiment of a second component 114 of a kit 110 according to the present invention in different perspective views. The second portion 210 is designed complementarily to the first portion 206 of the second component 114. Thus, reference is made to the description of FIGS. 4A and 4B above. The receptacles 214 may protrude from an outer surface 222 of the second component 114.

The second portion 210 may comprise at least one second half 220 of the receptacle 214 for one or more of the fiducials 216. In a closed state of the first portion 206 and the second portion 210 the fiducials may be enclosed by the first half 212 and the second half 220.

### LIST OF REFERENCE NUMBERS

110 kit
112 first component
114 second component
116 third component
118 non-invasive component
120 adhesive material
122 plaster
124 base part
126 arresting mechanism
128 front side
130 rear side
132 flat surface
134 direction of extension
136 snap-fit mechanism
138 receptacle
140 receptacle shape
142 surface
144 sliding direction
146 introducing end
148 opposing end
150 rectangular receptacle
152 channel-shaped receptacle
154 protrusion
156 receptacle
158 dovetail joint
160 dovetail guide
162 longitudinal protrusion
164 first end wall
166 front side
168 rear side
170 second counterpart
172 protrusion
174 protrusion shape
176 outer protrusion
178 intermediate protrusion
180 introducing intermediate protrusion end
182 snap-in hook
184 protrusion
188 opposing end
190 middle section
192 surface
194 dovetail groove
196 longitudinal slot
198 second end wall
200 tracking object
202 mounting unit
204 hole
206 first portion
208 first counterpart
210 second portion
212 first half
214 receptacle
216 fiducial
218 rear side
220 second half
222 outer surface

1. A kit for a fiducial marker-based registration of preinterventional image data to an intra-interventional scene, the kit comprising:
at least one first component, wherein the first component is configured for attachment to a body of a patient, wherein the first component comprises at least one base part of an arresting mechanism,
at least one second component, wherein the second component comprises at least one fiducial which is localizable in the preinterventional image data, wherein the second component comprises at least one first counterpart of the arresting mechanism,
at least one third component, wherein the third component comprises at least one mounting unit for mounting a spatially localizable tracking object on the third component, wherein the third component comprises at least one second counterpart of the arresting mechanism;
wherein the second component and the third component are selectively attachable to the first component by establishing a releasable mechanical connection between the base part and the first counterpart or between the base part and the second counterpart.

2. The kit according to claim 1, wherein the first component is a non-invasive component.

3. The kit according to claim 1, wherein the first component comprises at least one adhesive material attachable to a skin site of the patient.

4. The kit according to claim 1, wherein the kit comprises a single one of the first component, a single one of the second component and a single one of the third component.

5. The kit according to claim 1, wherein the arresting mechanism comprises a snap-fit mechanism.

6. The kit according to claim 1, wherein the arresting mechanism comprises a dovetail joint.

7. The kit according to claim 1, wherein the base part has at least one dovetail guide, wherein the dovetail guide is located at at least one first end wall of the first component, wherein the first counterpart and the second counterpart have a dovetail groove, wherein the dovetail groove is located at at least one second end wall of the second component and of the third component.

8. The kit according to claim 1, wherein the arresting mechanism comprises at least one locking mechanism, wherein the locking mechanism is configured to be manually activated and deactivated by a user.

9. The kit according to claim 1, wherein the second component and the third component are both configured to be attachable to the first component by moving the second component and the third component parallel to a skin site of the patient.

10. The kit according to claim 1, wherein the fiducial of the second component is exchangeable.

11. The kit according to claim 1, wherein the second component comprises at least one first portion and at least one second portion, wherein the first portion and the second portion are reversibly assemblable to each other, wherein the first portion comprises at least one first half of a receptacle for one of the fiducials, wherein the second portion comprises at least one second half of the receptacle for one of the fiducials, wherein the fiducials are exchangeable by disassembling the first portion and the second portion.

12. The kit according to claim 1, wherein the second component comprises a plurality of the fiducials, wherein the fiducials are located on different levels of the second component.

13. The kit according to claim 1, wherein the tracking object is selected from the group consisting of: an electromagnetic sensor; an electromagnetic field generator; a transponder; a sensor array; an optical sensor; active optical markers of optical tracking systems; passive optical markers of optical tracking systems; connectors for mechanical tracking; stereotactic devices; inertial tracking systems; ultrasound-based tracking devices; radio-frequency identification system.

14. A method for conducting a fiducial marker-based registration of preinterventional image data to an intra-interventional scene, the method comprising the following steps:
a) providing at least one kit according to claim 1;
b) attaching the first component to the body of the patient;
c) attaching the second component to the first component by establishing the releasable mechanical connection between the base part and the first counterpart;
d) acquiring preinterventional image data via the first component and the second component and localizing the fiducial of the second component in the preinterventional image data;
e) removing the second component from the first component by releasing the mechanical connection between the base part and the first counterpart;
f) mounting the tracking object onto the third component via the mounting unit;
g) attaching the third component to the first component by establishing a releasable mechanical connection between the base part and the second counterpart;
h) using information obtained from the first component, the second component and the third component and information obtained from medical imaging to generate supportive information for physicians during a medical intervention; and
i) removing the third component from the first component by releasing the mechanical connection between the base part and the second counterpart.

15. The method according to claim 14, wherein step h) comprises determining a position of a medical device relative to the tracking object thereby establishing the intra-interventional scene.
